(19) United States
(12) Patent Application Publication
(10) Pub. No.: US 2014/0221320 A1
(43) Pub. Date: Aug. 7, 2014

(54) TOPICAL MINOCYCLINE OINTMENT FOR SUPPRESSION OF ALLERGIC SKIN RESPONSES

(71) Applicants: Rauno Olev Joks, Port Washington, NY (US); Helen G. Durkin, Manhasset, NY (US)

(72) Inventors: Rauno Olev Joks, Port Washington, NY (US); Helen G. Durkin, Manhasset, NY (US)

(73) Assignee: THE RESEARCH FOUNDATION FOR THE STATE UNIVERSITY OF NEW YORK, Albany, NY (US)

(21) Appl. No.: 14/130,995

(22) PCT Filed: Jul. 6, 2012
(86) PCT No.: PCT/US2012/045645

§ 371 Date: Apr. 2, 2014

Related U.S. Application Data
(60) Provisional application No. 61/505,801, filed on Jul. 8, 2011.

(51) Int. Cl. A61K 9/00; A61K 31/65

(52) U.S. Cl. CPC A61K 9/0014; A61K 31/65
USPC 514/152

(57) ABSTRACT

The method of the present application is directed towards a method for suppressing an allergic response in response to an allergic trigger. This method comprises the following steps; applying, topically, to an affected area an effective amount of a minocycline composition so that the minocycline composition contacts the affected area for an effective amount of time and removing the minocycline composition from the affected area.

### FIELD OF THE DISCLOSURE

The disclosure relates generally to the field of topical administration of a minocycline ointment. More specifically, the present disclosure is directed to suppressing an allergic skin response on a subject's skin by administering an ointment comprising minocycline.

### BACKGROUND OF THE DISCLOSURE

An allergic reaction occurs because of a hypersensitivity of the immune system to an allergen. The reaction is characterized by excessive activation of mast cells and basophils which then can cause rashes, redness, itching and inflammation among other symptoms. For many allergic reactions of skin, the skin becomes reddened in a wheal pattern. A wheal is a rounded or flat-topped, pale red papule or plaque that is characteristically evanescent, typically disappearing within about 24 to about 48 hours.

Along with other characteristics, inflammation occurs in a majority of allergic reactions. While it is difficult to definitively describe inflammatory phenomena in terms of underlying cellular events, there are certain features that are generally agreed upon to be characteristic. These include fenestration of the micro-vasculature, leakage of the elements of blood into the interstitial spaces and migration of leukocytes into the inflamed tissue. On a macroscopic level, this is usually accompanied by the clinical signs of erythema, edema, tenderness and pain.

During this response, chemical mediators such as histamine, serotonin, leukotrienes, prostaglandins, various chemotactic factors, bradykinin, lymphokines, kinin and complement system, lysosomal enzymes and cyclic nucleotides are liberated locally. Phagocytic cells migrate into the area and cellular lysosomal membranes bay be ruptured, releasing lytic enzymes. All of these events contribute to the inflammatory response.

Topical drugs have been used to counteract the symptoms of an allergic reaction, including inflammation, such as hydrocortisone. Hydrocortisone is a steroid hormone with several risks including reduced bone formation, increased blood pressure thinning of skin and adrenal suppression.

What is desired is a topical treatment without the known drawbacks of many topical treatments presently available. Further, what is desired is a method of suppressing an allergic response by administering, topically, an ointment to limit mast cell mediated late phase allergic inflammation.

Embodiments of the present application provide a composition and method that addresses the above and other issues.

### SUMMARY OF THE DISCLOSURE

The method of the present application is directed towards a method for suppressing an allergic response in response to an allergic trigger. This method comprises the following steps; applying, topically, to an affected area an effective amount of a minocycline composition so that the minocycline composition contacts the affected area for an effective amount of time and removing the minocycline composition from the affected area.

### DETAILED DESCRIPTION

The present application is directed towards a method for suppressing an allergic response. This allergic response includes one or more of dermatitis, atopic dermatitis, urticaria, contact dermatitis, eczema, conjunctivitis and rhinoconjunctivitis. The allergic response is initially caused by an allergic trigger. The allergic trigger activates the mast cells and basophils and results in the known allergic symptoms of inflammation, etc. The allergic trigger can include any one or more triggers that result in an allergic response. These triggers include but are not limited to inhalation of environmental triggers, such as pollen, pet dander, mold etc., ingestion of dietary triggers such as shellfish, nuts, products containing gluten etc., ingestion or application of pharmaceutical triggers, such as penicillin, and pharmaceuticals containing sulfur, etc., and contact with triggers such as latex, poison ivy, poison oak, poison sumac and jewelry containing metals such as nickel, etc.

The present method of suppressing an allergic response caused by an allergic trigger includes topical application to an area affected by the allergic response an effective amount of a minocycline composition for an effective amount of time. The minocycline composition can be applied in any form, including as a liquid, gel, cream, lotion, paste, ointment, foam, spray, mist, aerosol and combinations of these forms. In the applied form, the minocycline can be present in any effective amount in the minocycline composition, including a concentration from about 0.1% to about 10% of the total weight of the composition.

The minocycline composition is topically applied so that the minocycline composition contacts the affected area for an effective amount of time. The effective amount of time can differ based on the allergic response to be suppressed and can range from between about 1 minute to about 48 hours.

To ensure that the minocycline composition contacts the affected area for the effective amount of time, the minocycline composition can be applied as a coating on or a filler in a dressing, a coating on or a filler in a substrate or a coating on or a filler in a patch. This administration will ensure that the minocycline composition remains in contact for the effective time and that the subject does not cause the minocycline composition to rub off or be washed off during the contact time. For example, if the subject is a human, and the minocycline composition is applied as a coating on a dressing, the dressing can remain on the affected area to guard the affected area from touching clothing or water during the duration of the contact time.

The affected area can be anywhere on the subjects body including the subject's skin, eyes, exposed mucosa. The exposed mucosa can be any mucosa, including oral, nasal, ano-genital and tympanic mucosa.

Following topical application of the minocycline for the effective amount of time, the minocycline composition is removed from the affected area. Removal can include physical removal by wiping or scrubbing or similar actions, removal can include absorption through the skin. At this point, the allergic reaction has been suppressed and the typical symptoms of the allergic reaction, including inflammation etc., have been reduced as compared to non-treatment with the minocycline composition.

This cycle of application and removal can be repeated 2 or more times if desired.

### EXAMPLE 1

A topical minocycline ointment was prepared by mixing 600 mg of minocycline with 30 grams of an Aquaphor® ointment to produce a 2% minocycline ointment. Eight adult human subjects with known respiratory allergies to pollen, molds or dander were tested. A skin prick test (Dermapik®) to four aeroallergens was performed on both sides of the upper back of each of the subjects. Wheal size was measured at 15 minutes, 1 hour and 24 hours for each side. On one side of the upper back of each subject, minocycline ointment was placed on the skin and covered with an adherent dressing for a total of 48 hours. The other side of the upper back of each subject was left untreated and was not dressed or bandaged.

Mean diameters of the wheals for each side of each subject's back were calculated and skin responses were evaluated with respect to current QOL (Juniper) scores. For statistical analysis, a mixed linear model was constructed, with dependent variable mean wheal diameter (square-root transformed, to preserve symmetry and homogeneity of variance).

A significant time-by-minocycline interaction was detected (F[2.82]=5.87, p=0.004). Effects analysis showed significant differences between minocycline conditions at 24 hours (F(1.66)=8.88, p=0.004), but not at 15 minutes (F[1.43]=3.95, p=0.053) or at 1 hour (F[1.54]=0.20, p=0.654). No significant three-way interaction involving allergen, Rhinoconjunctivitis Quality of Life Questionnaire (RQLQ) or Asthma Quality of Life Questionnaire (AQLQ) were detected.

The presence of minocycline significantly reduced late phase mean wheal diameter 24 hours after allergen administration. No significant differential effects of minocycline were found across allergens. No significant differential effects of minocycline were found depending on subject RQLQ or AQLQ scores.

What is claimed is:

1. A method for suppressing an allergic response in response to an allergic trigger in an affected area, the method comprising:
applying, topically, to the affected area an effective amount of a minocycline composition so that the minocycline composition contacts the affected area for an effective amount of time, and
removing the minocycline composition from the affected area.

2. The method of claim 1, wherein minocycline is present in the minocycline composition at a concentration from about 0.1% to about 10%.

3. The method of claim 1, wherein the effective amount of time is from about 1 minute to about 48 hours.

4. The method of claim 1, wherein the composition is selected from the group consisting of liquids, gels, creams, lotions, pastes, ointments, foams, sprays, mists, aerosols and combinations thereof.

5. The method of claim 1, wherein the composition is applied as one of the group selected from a coating on a dressing, a filler in a dressing, a coating on a substrate, a filler in a substrate, a coating in a patch and a filler in a patch.

6. The method of claim 1, wherein the affected area is selected from the group consisting of skin, eyes, exposed mucosa and combinations thereof.

7. The method of claim 6, wherein the mucosa is selected from the group consisting of oral, nasal, ano-genital, tympanic and combinations thereof.

8. The method of claim 1, wherein the subject is a human.

9. The method of claim 1, wherein the applying and removing steps are repeated.

10. The method of claim 1, wherein the allergic response of the affected area is selected from the group consisting of dermatitis, atopic dermatitis, urticaria, contact dermatitis, eczema, conjunctivitis, rhinoconjunctivitis and combinations thereof.

11. The method of claim 1, wherein the allergic trigger is selected from the group consisting of an environmental trigger, a dietary trigger, a pharmaceutical trigger, a contact trigger.

12. The method of claim 11, wherein the environmental trigger is selected from the group consisting of pollen, pet dander and mold.

13. The method of claim 11, wherein the dietary trigger is selected from the group consisting of shellfish, nuts and gluten containing products

14. The method of claim 11, wherein the pharmaceutical trigger is selected from the group consisting of penicillin and pharmaceuticals comprising sulfur.

15. The method of claim 11, wherein the contact trigger is selected from the group consisting of latex, poison ivy, poison oak, poison sumac, metal jewelry and combinations thereof.
